(19) United States
(12) Patent Application Publication
(10) Pub. No.: US 2020/0289146 A1
(43) Pub. Date: Sep. 17, 2020

(54) ROTATING SCALPET INCLUDING OVERMOLDED GEARS, AND METHOD FOR MANUFACTURING AND USING SAME

(71) Applicant: Recros Medica, Inc., San Diego, CA (US)

(72) Inventors: Randy Jordheim, Dublin, CA (US); Marlo Cinco, Danville, CA (US); Brett Isakovic, San Jose, CA (US); Francisco Magno, San Roman, CA (US); Jerome Adam-Cote, San Francisco, CA (US); James McCrea, San Carlos, CA (US); Edward W. Knowlton, Reno, NV (US)

(21) Appl. No.: 16/800,555

(22) Filed: Feb. 25, 2020

Related U.S. Application Data
(60) Provisional application No. 62/816,650, filed on Mar. 11, 2019.
(60) Provisional application No. 62/816,650, filed on Mar. 11, 2019.

(51) Int. Cl. A61B 17/3205; A61B 90/00

(52) U.S. Cl. CPC A61B 17/32053; A61B 2017/00747; A61B 90/03

(57) ABSTRACT

A rotatable scalpet for rotational fractional resection, and method of manufacturing same, is provided. In an embodiment, a rotatable scalpet includes a hollow tube extending along a longitudinal axis and including an inner surface and an outer surface, the inner surface forming a passageway between a first end and a second end of the hollow tube, a cutting edge located at the first end of the hollow tube, the cutting edge configured to cut a patient's tissue when the hollow tube is rotated around the longitudinal axis, and a gear molding feature formed into the outer surface of the hollow tube, the gear molding feature enabling a gear to be molded around at least a portion of the hollow tube.

### PRIORITY CLAIM

The present application claims priority to and the benefit of U.S. Provisional Patent Application No. 62/816,650, filed on Mar. 11, 2019, the entirety of which is incorporated herein by reference.

### TECHNICAL FIELD

The present disclosure generally relates to a scalpet with an overmolded gear and a method of manufacturing same, and more specifically to a rotatable scalpet with the gear molded into the outer surface of the scalpet.

### BACKGROUND

Rotational fractional resection (“RFR”) is a procedure which may be used to achieve focal aesthetic contouring by removing lax skin and excess fat tissue from a patient. Skin may be removed by the use of a rotating scalpet, which is a hollow, sharpened tube which excises full thickness dermal resections. Due to the presence of longitudinal and/or rotational forces, however, the scalpet may fail if not properly formed.

### SUMMARY

The present disclosure provides an improved scalpet that can withstand increased longitudinal and/or rotational forces, and a method of forming same. In a general example embodiment, a rotatable scalpet includes a hollow tube extending along a longitudinal axis and including an inner surface and an outer surface, the inner surface forming a passageway between a first end and a second end of the hollow tube, a cutting edge located at the first end of the hollow tube, the cutting edge configured to cut a patient's tissue when the hollow tube is rotated around the longitudinal axis, and a gear molding feature formed into the outer surface of the hollow tube, the gear molding feature enabling a gear to be molded around at least a portion of the hollow tube.

In another embodiment, the gear molding feature includes at least one aperture extending through the outer surface of the hollow tube to the passageway.

In another embodiment, the gear molding feature includes at least one indentation extending into the outer surface of the hollow tube, wherein a depth of the at least one indentation is less than a thickness of the outer tube.

In another embodiment, the gear molding feature includes a plurality of apertures.

In another embodiment, the plurality of apertures are aligned in a row along the longitudinal axis of the hollow tube.

In another embodiment, the plurality of apertures are aligned at a same height along the longitudinal axis of the hollow tube.

In another embodiment, the gear molding feature includes at least one slot.

In another embodiment, the at least one slot extends parallel to the longitudinal axis.

In another embodiment, the at least one slot includes a radial indentation extending perpendicular to the longitudinal axis around a diameter of the outer surface.

In another embodiment, the gear molding feature includes a plurality of slots aligned at a same height along the longitudinal axis of the hollow tube.

In another embodiment, the gear molding feature includes at least one radial indentation creating a knurled surface on a portion of the outer surface of the hollow tube.

In another embodiment, the rotatable scalpet includes the gear molded into the gear molding feature.

In another general example embodiment, a rotatable scalpet includes a hollow tube extending along a longitudinal axis and including an inner surface and an outer surface, the inner surface forming a passageway between a first end and a second end of the hollow tube, a cutting edge located at the first end of the hollow tube, the cutting edge configured to cut a patient's tissue when the hollow tube is rotated around the longitudinal axis, and a gear configured to rotate the scalpet, the gear molded into the outer surface of the hollow tube.

In another embodiment, the gear is molded into at least one aperture extending through the outer surface of the hollow tube.

In another embodiment, the gear is molded into an indentation into the outer surface of the hollow tube, wherein a depth of the at least one indentation is less than a thickness of the outer tube.

In another embodiment, a rotational fractional resection device includes the rotating scalpet.

In another general example embodiment, a method of manufacturing a rotatable scalpet includes providing a hollow tube extending along a longitudinal axis and including an inner surface and an outer surface, the inner surface forming a passageway between a first end and a second end of the hollow tube, forming a gear molding feature into the outer surface of a hollow tube, and molding a gear over the gear molding feature by dispensing at least a portion of a material used to form the gear into the gear molding feature.

In another embodiment, molding the gear includes dispensing the material used to form the gear into an aperture of the gear molding feature.

In another embodiment, molding the gear includes dispensing the material used to form the gear into an indentation of the gear molding feature.

In another embodiment, the method includes curing the molded gear to harden the molded gear within the gear molding feature.

In another embodiment, the entire scalpet with the gear is an injection molded part.

In another embodiment, the entire scalpet with the gear is a 3D printed part.

In another embodiment, the entire scalpet with the gear is a molten metal 3D printed part.

The advantages discussed herein may be found in one, or some, and perhaps not all of the embodiments disclosed herein. Additional features and advantages are described herein, and will be apparent from the following Detailed Description and the figures.

### BRIEF DESCRIPTION OF THE FIGURES

FIG. 1 shows a perspective view of an example embodiment of a rotatable scalpet in accordance with the present disclosure.

FIG. 2 shows a side view of an example embodiment of a hollow tub which may be used to form the scalpet of FIG. 1.

FIG. 3 shows a cross-sectional view taken vertically through the center of the hollow tube of FIG. 2, with a gear molded to the outer surface of the hollow tube.

FIGS. 4A to 4C show cross-sectional views which may be taken horizontally through the hollow tube of FIG. 2 at the locations of the apertures therein.

FIG. 5 shows a side view of an example embodiment of a hollow tub which may be used to form the scalpet of FIG. 1.

FIG. 6 shows a cross-sectional view taken vertically through the center of a the hollow tube of FIG. 5, with a gear molded to the outer surface of the hollow tube.

FIGS. 7A to 7C show cross-sectional views which may be taken horizontally through the hollow tube of FIG. 8 at the locations of the slots therein.

FIG. 8 shows a side view of an example embodiment of a hollow tub which may be used to form the scalpet of FIG. 1.

FIG. 9 shows a cross-sectional view taken vertically through the center of the hollow tube of FIG. 8, with a gear molded to the outer surface of the hollow tube.

FIG. 10 shows an example embodiment of a method of manufacturing the scalpet of FIG. 1.

FIG. 11 shows an example embodiment of a device using a plurality of the scalpets of FIG. 1.

### DETAILED DESCRIPTION

Before the disclosure is described, it is to be understood that this disclosure is not limited to the particular apparatuses and methods described. It is also to be understood that the terminology used herein is for the purpose of describing particular embodiments only, and is not intended to be limiting, since the scope of the present disclosure will be limited only to the appended claims.

As used in this disclosure and the appended claims, the singular forms “a,” “an” and “the” include plural referents unless the context clearly dictates otherwise. The methods and apparatuses disclosed herein may lack any element that is not specifically disclosed herein. Thus, “comprising,” as used herein, includes “consisting essentially of” and “consisting of.”

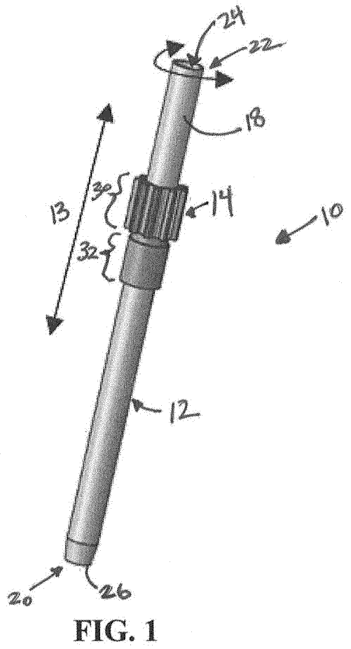

The present disclosure is directed to a scalpet that may be used, for example, by a rotational rotational fractional resection (“RFR”) device, either alone or in combination with other scalpets having similar or the same features. FIG. 1 illustrates an example embodiment of a scalpet 10 according to the present disclosure. In the illustrated embodiment, scalpet 10 includes a hollow tube 12 and a gear 14 encircling the hollow tube 12. In use, the gear 14 may be used to rotate the hollow tube 12 and/or to cause another scalpet to rotate using the motion of the hollow tube 12.

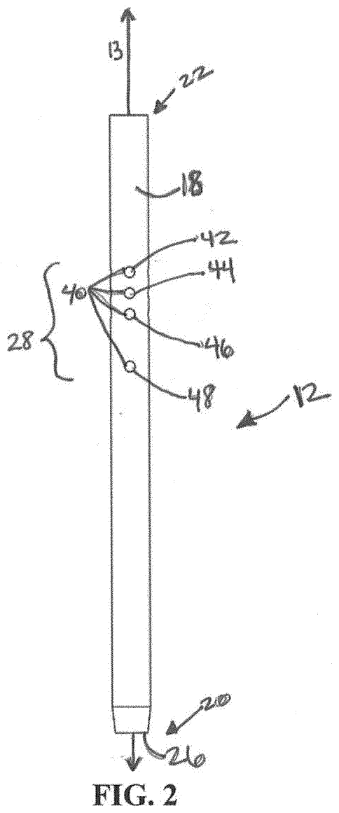

Hollow tube 12 includes an inner surface 16 and an outer surface 18 extending between a first end 20 and a second end 22. Inner surface 16 forms a passageway 24 through the hollow tube 12, along the longitudinal axis 13 between the first end 20 and the second end 22, placing the opening at the first end 20 in communication with the opening at the second end 22. Outer surface 18 includes a gear molding feature 28 (shown in FIG. 2), which enables the gear 14 to be molded thereto, as explained in more detail below.

At the first end 20 of the hollow tube 12, the scalpet 10 includes a beveled cutting edge 26 which is configured to cut into a patient's tissue when the scalpet 10 is rotated, for example, via the gear 14. At a second end 22 of the hollow tube 12, a vacuum may be applied for collecting incised tissue. By rotating the cutting edge 26 into a patient's tissue using the gear 14, and then applying a vacuum at second end 22, the patient's tissue that is cut by the cutting edge 26 may be removed from the patient via passageway 24. The scalpet 10 may be configured to provide a tissue resection density between 10% and 25%.

In an embodiment, the hollow tube 12 may be formed of metal, for example, SAE 304 stainless steel, SAE 316 stainless steel, or the like. In the illustrated embodiment, the hollow tube 12 is about 29.5 mm in length along the longitudinal axis 13, the passageway 24 is 1.5 mm in diameter, and the hollow tube 12 is about 1.83 mm thick, but those of ordinary skill in the art will recognize that other dimensions may be used. For example, the hollow tube 12 may have a diameter between 0.8 mm and 1.2 mm.

In the illustrated embodiment, the gear 14 is molded to the hollow tube 12 so as to encircle the hollow tube 12 at a location between the first end 20 and the second end 22 along the longitudinal axis 13. In the illustrated embodiment, the gear 14 is located closer to the second end 22 than to the first end 20, but those of ordinary skill in the art will recognize that other configurations are possible. In the illustrated embodiment, toothed gear portion 30 is located about 7 mm from the second end 22, which has been determined to be effective for desired cutting depths into the patient for this design, but those of ordinary skill in the art will recognize that other configurations are possible.

In the illustrated embodiment, the gear 14 includes the toothed gear portion 30 and a smooth portion 32. The toothed gear portion 30 may be used, for example, to communicate with other gears to cause rotation of the scalpet 10 and/or additional scalpets or other elements. The smooth portion 32 adds strength to the securement of the gear 14 to the outer surface 18 by adding to the overall length of the gear 14 while minimizing the size of the teeth extending therefrom. The smooth portion 32 is also advantageous because it serves as a location for tooling to inject the plastic material for the gear 14 without distorting the teeth of the toothed gear portion 30 during manufacturing. Those of ordinary skill in the art will recognize that the sizes of the toothed gear portion 30 and the smooth portion 32 may be altered, or smooth portion may be eliminated and toothed portion may extend the entire length of the gear 14.

In an embodiment, the gear 14 is molded with plastic, but those of ordinary skill in the art will recognize that other materials may be used.

FIGS. 2 to 9 illustrate example embodiments of the gear molding features 28 which may be used to attach the gear 14 to the hollow tube 12, for example, by the molding gear 14 into the outer surface 18 of the hollow tube 12. As explained in more detail below, one or more gear molding features 28 may enable more material of the gear 14 to adhere to the outer surface 18 and more securely attach to the hollow tube 12 in comparison with alternative designs, enabling more torque to be applied to the gear 14 without causing the scalpet 10 to break or fail. In other words, the gear molding features 28 enables the gear 14 to be attached with additional strength to keep the gear 14 on or otherwise attached to the hollow tube 12 in the presence of longitudinal and/or rotational forces. It has been found, for example, that longitudinal strength in particular is improved with this design.

FIGS. 2 to 4 show a first embodiment of a gear molding feature 28. In FIGS. 2 to 4, the gear molding feature 28 includes at least one aperture 40 extending through the outer surface 18 to the inner surface 16 and the passageway 24. More specifically, the at least one aperture 40 includes a plurality of apertures 40 comprising a plurality of first apertures 42 at a first longitudinal location along the longitudinal axis 13, a plurality of second apertures 44 at a second longitudinal location along the longitudinal axis 13m a plurality of third apertures 46 at a third longitudinal location along the longitudinal axis 13, and/or a plurality of fourth apertures 48 at a fourth longitudinal location along the longitudinal axis 13. As illustrated, the first, second and third longitudinal locations are spaced at equal distances, whereas the gap between the third and fourth longitudinal locations is greater than the gap between the first and second longitudinal locations or the second and third longitudinal locations. In the illustrated embodiment, the top three apertures are evenly spaced to provide uniform adherence directly under the toothed gear portion 30, while the fourth aperture is located underneath the smooth portion 32 which carries less load and requires less adherence. In the illustrated embodiment, a plurality of apertures 40 are aligned in a row along the longitudinal axis 13 at multiple locations around the hollow tube 12, but it should be understood that the apertures may also be offset along the longitudinal axis 13. Those of ordinary skill in the art will recognize alternative configurations which may be used.

In the illustrated embodiment, the plurality of apertures 40 are between about 0.4 mm and 0.6 mm in diameter, and are spaced apart by about 0.8 mm to about 1.2 mm parallel to the longitudinal axis 13. Those of ordinary skill in the art will recognize that other dimensions may be used.

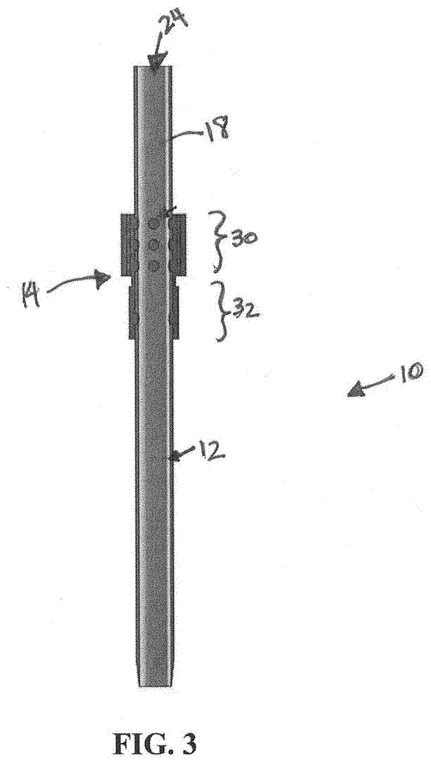

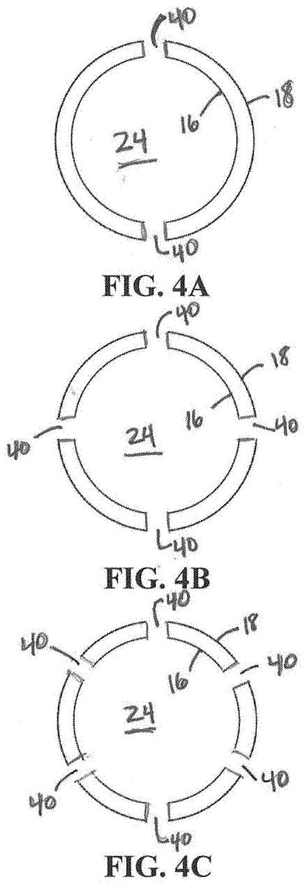

FIGS. 4A to 4C show a cross section of the hollow tube 12, demonstrating example embodiments of how any of the apertures 40 may be positioned around the hollow tube 12. In FIG. 4A, two apertures 40 are placed on opposing sides of the hollow tube 12. In FIG. 4B, four apertures 40 are equally spaced in quarter sections around the hollow tube 12. In FIG. 4C, six apertures 40 are equally spaced in sixth sections around the hollow tube 12. Those of ordinary skill in the art will understand that different configurations may be used, and that the more apertures 40 used, the stronger the attachment between the gear 14 and the hollow tube 12, particularly when torque is applied to rotate the scalpet 10. In FIGS. 2 and 3, the first apertures 42, second apertures 44 and third apertures 46 are positioned as shown in FIG. 4B, while the fourth apertures 48 are positioned as shown in FIG. 4A.

As illustrated in FIG. 3, the gear 14 is molded into the plurality of apertures 40 of the gear molding feature 28. The gear 14 may be molded to the hollow tube 12, for example, by surrounding the hollow tube 12 with a mold having the appropriate shape of the gear 14, and then injecting liquid plastic or another material for the gear 14 into the mold. As liquid material is injected into the mold, the material migrates into the plurality of apertures 40, causing the plurality of apertures 40 to fill with the same material used to mold the gear 14, increasing the adherence strength of the gear 14 once the liquid material hardens. A pin may be inserted into the hollow tube 12 to prevent the liquid plastic from flowing into the hollow tube 12. If plastic forms inside the passageway 24, it may prevent effective removal of tissue that is pulled through passageway 24 with a vacuum.

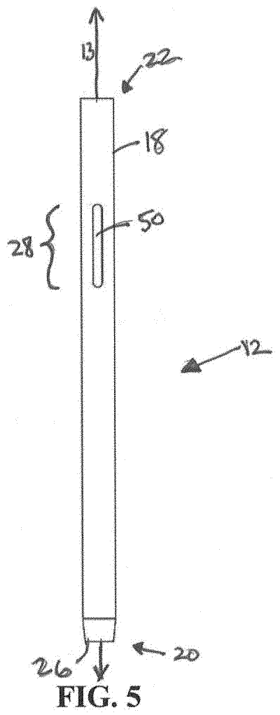

FIGS. 5 to 7 show a second embodiment of a gear molding feature 28. In FIGS. 5 to 7, the gear molding feature 28 includes at least one longitudinal slot 50 extending through the outer surface 18 to the inner surface 16. More specifically, the at least one longitudinal slot 50 includes a plurality of longitudinal slots 50 extending parallel to the longitudinal axis 13. In the illustrated embodiment, each slot 50 is between about 0.4 mm and 0.6 mm in width perpendicular to the longitudinal axis 13, and is between about 0.8 mm to about 1.2 mm in length parallel to the longitudinal axis 13. Those of ordinary skill in the art will recognize that other dimensions may be used.

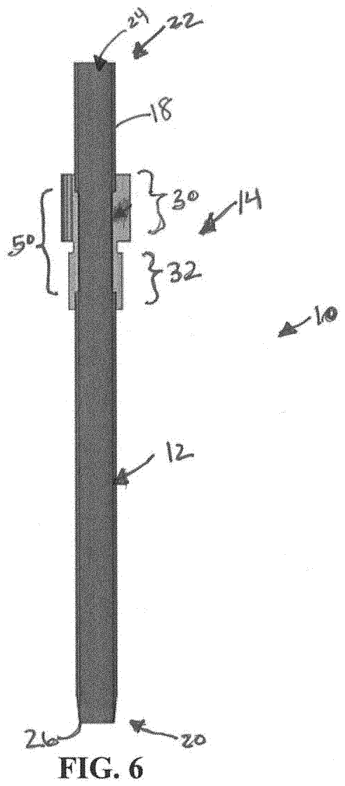

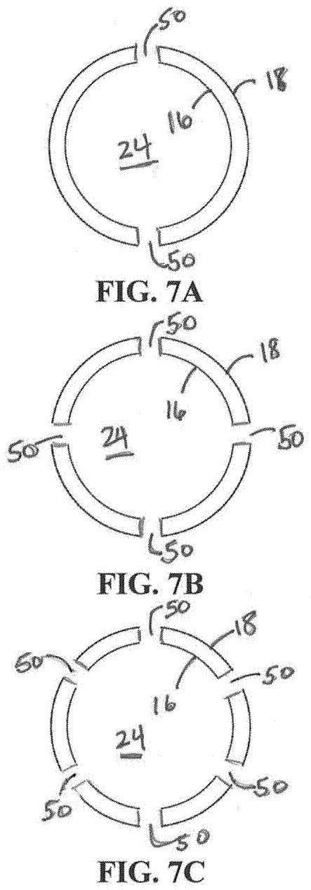

FIGS. 7A to 7C show a cross section of the hollow tube 12, demonstrating example embodiments of how any of the longitudinal slots 50 may be positioned around the hollow tube 12. In FIG. 7A, two slots 50 are placed on opposing sides of the hollow tube 12. In FIG. 7B, four longitudinal slots 50 are equally spaced in quarter sections around the hollow tube 12. In FIG. 7C, six longitudinal slots 50 are equally spaced in sixth sections around the hollow tube 12. In FIGS. 5 and 6, the longitudinal slots 50 are positioned as shown in FIG. 7A.

As illustrated in FIG. 6, the gear 14 is molded into slots 50 of gear molding feature 28. The gear 14 may be molded to the hollow tube 12, for example, by surrounding the hollow tube 12 with a mold having the appropriate shape of the gear 14, and then injecting liquid plastic or another material for the gear 14 into the mold. As liquid material is injected into the mold, it migrates into the slots 50, causing the slots 50 to fill with the same material used to mold the gear 14, increasing the adherence strength of the gear 14 once the liquid material hardens. A pin may be inserted into the hollow tube 12 to prevent the liquid plastic from flowing into the hollow tube 12. If plastic forms inside the passageway 24, it may prevent effective removal of tissue that is pulled through the passageway 24 with vacuum.

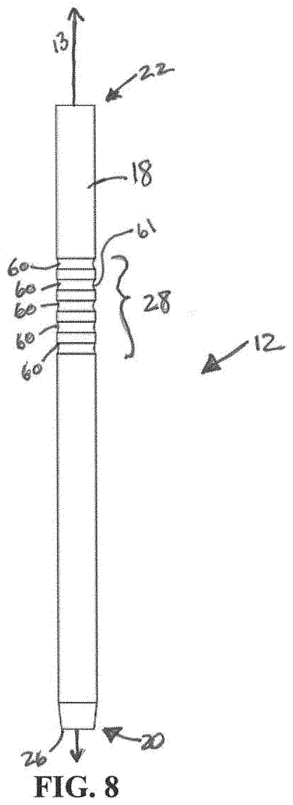

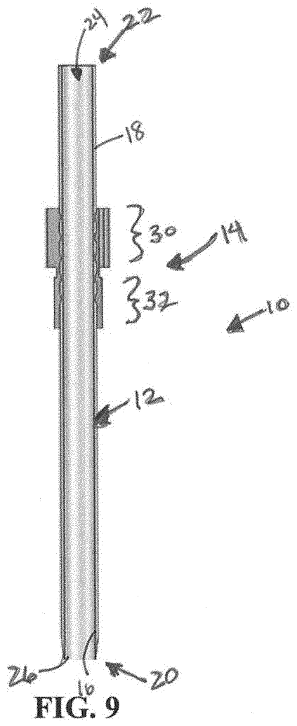

FIGS. 8 and 9 show a third embodiment of a gear molding feature 28. In FIGS. 8 and 9, the gear molding feature 28 includes a knurled surface 61 created by at least one radial indention 60 extending into the outer surface 18, wherein the depth of the at least one radial indentation 60 is less than the thickness of the hollow tube 12 between the inner surface 16 and the outer surface 18, with the at least one radial indentation 60 not passing through the entire thickness of the hollow tube 12 to the passageway 24. In an embodiment, the at least one radial indentation extends into the outer surface 18 about 50% or less of the entire thickness of the hollow tube 12 between the inner surface 16 and the outer surface 18. In the illustrated embodiment, five radial indentations 60 are shown, but those of ordinary skill in the art will recognize that more or less may be used. Additionally, the illustrated embodiment shows each radial indentation 60 encircling the entirety of the hollow tube 12, but those of ordinary skill in the art will recognize that the radial indentions may instead only partially encircle the hollow tube 12, for example, by extending about 25%, 50% and/or 75% around the outer surface of the hollow tube 12.

In the illustrated embodiment, the radial indentations 60 are each between 0.4 mm and 0.6 mm in width parallel to longitudinal axis 13, and are spaced apart by 0.8 mm to 1.2 mm parallel to longitudinal axis 13. In the illustrated embodiment, the radial indentations are equidistant from each other, but they may also be spaced at varying distances. Those of ordinary skill in the art will recognize alternative configurations which may be used.

As illustrated in FIG. 9, the gear 14 is molded into the radial indentations 60 of the knurled surface 61 of the gear molding feature 28. The gear 14 may be molded to the hollow tube 12, for example, by surrounding the hollow tube 12 with a mold having the appropriate shape of the gear 14, and then injecting liquid plastic or another material for the gear 14 into the mold. As liquid material is injected into the mold, it migrates into the radial indentations 60 of the knurled surface 61, causing the radial indentations 60 to fill with the same material used to mold the gear 14, thereby increasing the adherence strength of the gear 14 once the liquid material hardens. A pin may be inserted into the hollow tube 12 to prevent the liquid plastic from flowing into the hollow tube 12. If plastic forms inside the passageway 24, it may prevent effective removal of tissue that is pulled through the passageway 24 with vacuum.

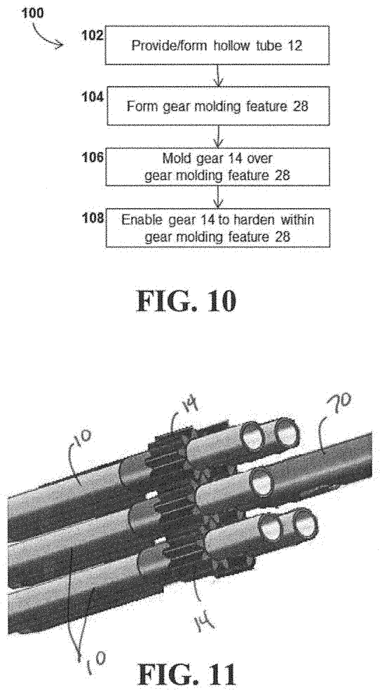

FIG. 10 illustrates a method 100 of manufacturing a scalpet 10 as illustrated in FIGS. 1 to 9. Those of ordinary skill in the art will recognize that additional steps may be added, or the illustrated steps may be omitted in some circumstances.

At step 102, a hollow tube 12 is obtained. The hollow tube 12 may be machined to create a passageway 24 and/or the cutting edge 26, or these elements may already be provided with the hollow tube 12. The hollow tube 12 may be formed of metal, for example, SAE 304 stainless steel tubing, which may be formed, for example, by extruding the metal through a die or by welding a flat piece of metal into a tube. In an embodiment, the hollow tube 12 may be formed to be about 29.5 mm along its longitudinal axis 13, and may be formed with a passageway 24 of about 1.5 mm in diameter and a hollow tube 12 thickness of about 1.83 mm, but those of ordinary skill in the art will recognize that other dimensions may be used. In an embodiment, the hollow tube 12 may be provided without additional machining necessary to complete step 102. In an embodiment, hollow tube 12 may be an injection molded part. In an embodiment, the hollow tube 12 may be 3D printed, for example, using plastic or metal, for example, molten metal.

At step 104, at least one gear molding feature 28 is machined into hollow tube 12. The gear molding feature 28 may include, for example, an aperture 40, slot 50 and/or indentation 60 discussed above. The gear molding feature 28 may be machined into hollow tube 12, for example, by drilling partially or fully into the outer surface 18 of hollow tube, for example, at an angle perpendicular to the longitudinal axis 13.

At step 106, gear 14 is molded to hollow tube 12 at gear molding feature 28. Gear 14 may be molded to hollow tube 12, for example, by surrounding hollow tube 12 with a mold having the appropriate shape of gear 14, and then injecting liquid plastic or another material for gear 14 into the mold. As liquid material is injected into the mold, it migrates into the gear molding feature 28, causing the gear molding feature 28 to fill with the same material used to mold the gear 14, increasing the adherence strength of the gear 14 under longitudinal and/or rotational forces. A pin may be inserted into the hollow tube 12 during injection to prevent liquid plastic from adhering to the inner surface of the tube.

At step 108, the plastic to form the gear 14 hardens. In an embodiment, the plastic for the gear 14 may be injected at high pressure and temperature so that it cools in an ambient environment to form gear 14. In an alternative embodiment, the liquid material for gear 14 may be cured, for example, by applying heat or light to the material. Upon curing, the liquid material may harden, creating portions of gear 14 which project into outer surface 18 of hollow tube 12, which strengthen the bond between hollow tube 12 and gear 14, increasing the resistance of gear 14 to longitudinal and/or rotational forces.

In an alternative embodiment, the entire scalpet 10 may be an injection molded part. In an alternative embodiment, the entire scalpet 10 may be 3D printed, for example, using plastic or metal, for example, molten metal.

FIG. 11 illustrates an example embodiment of a device including plurality of scalpets 10 arranged with interacting gears 14 which cause the scalpets 10 to rotate simultaneously. In the illustrated embodiment, a central rod or tube 70 rotates a plurality of scalpets 10. In another embodiment, a gearing mechanism may rotate one or more scalpet 10, which may simultaneously cause additional scalpets 10 or other elements to rotate. In an embodiment, the device shown in FIG. 11 may be used in a rotational fractional resection (“RFR”) device. Those of ordinary skill in the art will recognize additional methods to utilize the present disclosure.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

We claim:

1: A rotatable scalpet comprising:
a hollow tube extending along a longitudinal axis and including an inner surface and an outer surface, the inner surface forming a passageway between a first end and a second end of the hollow tube;
a cutting edge located at the first end of the hollow tube, the cutting edge configured to cut a patient's tissue when the hollow tube is rotated around the longitudinal axis; and
a gear molding feature formed into the outer surface of the hollow tube, the gear molding feature enabling a gear to be molded around at least a portion of the hollow tube.

2: The rotatable scalpet of claim 1, wherein the gear molding feature includes at least one aperture extending through the outer surface of the hollow tube to the passageway.

3: The rotatable scalpet of claim 1, wherein the gear molding feature includes at least one indentation extending into the outer surface of the hollow tube, wherein a depth of the at least one indentation is less than a thickness of the outer tube.

4: The rotatable scalpet of claim 1, wherein the gear molding feature includes a plurality of apertures.

5: The rotatable scalpet of claim 4, wherein the plurality of apertures are aligned in a row along the longitudinal axis of the hollow tube.

6: The rotatable scalpet of claim 4, wherein the plurality of apertures are aligned at a same height along the longitudinal axis of the hollow tube.

7: The rotatable scalpet of claim 1, wherein the gear molding feature includes at least one slot.

8: The rotatable scalpet of claim 7, wherein the at least one slot extends parallel to the longitudinal axis.

9: The rotatable scalpet of claim 7, wherein the at least one slot includes a radial indentation extending perpendicular to the longitudinal axis around a diameter of the outer surface.

10: The rotatable scalpet of claim 1, wherein the gear molding feature includes a plurality of slots aligned at a same height along the longitudinal axis of the hollow tube.

11: The rotatable scalpet of claim 7, wherein the gear molding feature includes at least one radial indentation creating a knurled surface on a portion of the outer surface of the hollow tube.

12: The rotatable scalpet of claim 1, which includes the gear molded into the gear molding feature.

13: A rotatable scalpet comprising:
a hollow tube extending along a longitudinal axis and including an inner surface and an outer surface, the inner surface forming a passageway between a first end and a second end of the hollow tube;
a cutting edge located at the first end of the hollow tube, the cutting edge configured to cut a patient's tissue when the hollow tube is rotated around the longitudinal axis; and
a gear configured to rotate the scalpet, the gear molded into the outer surface of the hollow tube.

14: The rotating scalpet of claim 13, wherein the gear is molded into at least one aperture extending through the outer surface of the hollow tube.

15: The rotating scalpet of claim 13, wherein the gear is molded into an indentation into the outer surface of the hollow tube, wherein a depth of the at least one indentation is less than a thickness of the outer tube.

16: A rotational fractional resection device including the rotating scalpet of claim 13.

17: A method of manufacturing a rotatable scalpet, the method comprising:
providing a hollow tube extending along a longitudinal axis and including an inner surface and an outer surface, the inner surface forming a passageway between a first end and a second end of the hollow tube;
forming a gear molding feature into the outer surface of a hollow tube; and
molding a gear over the gear molding feature by dispensing at least a portion of a material used to form the gear into the gear molding feature.

18: The method of claim 17, wherein molding the gear includes dispensing the material used to form the gear into an aperture of the gear molding feature.

19: The method of claim 17, wherein molding the gear includes dispensing the material used to form the gear into an indentation of the gear molding feature.

20: The method of claim 17, which includes curing the molded gear to harden the molded gear within the gear molding feature.
